(19) United States
(12) Patent Application Publication
(10) Pub. No.: US 2013/0345388 A1
(43) Pub. Date: Dec. 26, 2013

(54) Process for Producing Nitrile-Fatty Acid Compounds

(71) Applicants: Markus Brandhorst, Lyon (FR); Jean-Luc Couturier, Lyon (FR); Jean-Luc Dubois, Millery (FR)

(72) Inventors: Markus Brandhorst, Lyon (FR); Jean-Luc Couturier, Lyon (FR); Jean-Luc Dubois, Millery (FR)

(73) Assignee: ARKEMA FRANCE, Colombes (FR)

(21) Appl. No.: 13/976,619

(22) PCT Filed: Dec. 14, 2011
(86) PCT No.: PCT/FR2011/052990

§ 371 Date: Sep. 6, 2013

(30) Foreign Application Priority Data
Jan. 10, 2011 (FR) 1150173
Jun. 14, 2011 (FR) 1155174

(51) Int. Cl. C07C 253/30

(52) U.S. Cl. CPC C07C 253/30
USPC 528/362; 554/113; 554/114

(57) ABSTRACT

The invention relates to a process for synthesizing a nitrile-fatty acid (heminitrile) from unsaturated fatty acids, in the form of an acid or a simple ester or a “complex” ester of triglyceride type, which is first of all converted into an unsaturated fatty nitrile which is subjected to oxidative cleavage using H₂O₂ as oxidizing agent. This process can be used for preparing polyamide monomers, such as ω-amino acids or diamines or diacids equivalent to said heminitrile and for obtaining polyamides from raw materials which are of natural origin and from a renewable source.

The work which led to this invention received financial support from the European Union in the context of Framework Program 7 (FP7/2007-2013) under project No. 241718 EUROBIOREF.

The invention relates to a process for synthesizing a nitrile-fatty acid, also referred to as heminitrile hereinafter, from unsaturated fatty acids, in the form of an acid or a simple ester or a “complex” ester of triglyceride type, which is first of all converted into an unsaturated fatty nitrile which is subjected to oxidative cleavage using H₂O₂ as oxidizing agent.

The nitrile-fatty acids of general formula NC—(CH₂)ₙ—COOH or of formula NC(CH₂)ₙ(CH═CH)ₘCOOH (empirical formula Cₙ₊₂ₘ₊₂H₂ₙ₊₂ₘ₊₁NO₂) in the case where the cleavage is carried out on a polyunsaturated acid, subsequently referred to as diacid heminitriles or more simply heminitriles, are intermediate compounds which can be used in the synthesis of an entire range of “fatty” compounds such as ω-amino acids, α-ω-dinitriles, α-ω-diamines or α-ω-diacids. The term “nitrile-fatty acid” is intended to mean linear nitrile-acid compounds having from 6 to 15 carbon atoms.

Current developments in environmental matters are leading, in the fields of energy and chemistry, to the exploitation of natural raw materials originating from a renewable source being favored. This is the reason why some studies have been undertaken to develop, on the industrial scale, processes using fatty acids/esters (vegetable oils or animal fats) as raw material for the manufacture of these fatty compounds, which can for example be used as polymerization monomers for obtaining a polyamide.

There is an abundant literature on the synthesis of various difunctional α-ω compounds from unsaturated natural fatty acids. This literature is particularly focused on “natural” oleic acid for the manufacture of ω-amino acids, such as 9-aminononanoic acid, which is the precursor for the synthesis of Nylon 9. It should in fact be recalled that the polyamide industry uses an entire range of monomers consisting of long-chain ω-amino acids, usually called Nylon, characterized by the length of the methylene chain (—CH₂)ₙ separating two amide —CO—NH— functions. Thus it is that Nylon 6 (based on 6-aminohexanoic acid), Nylon 7, Nylon 8, Nylon 9, Nylon 11, Nylon 13, and the like, are known.

The main studies have related to the synthesis of 9-aminononanoic acid, which is the precursor of Nylon 9, from oleic acid of natural origin. With regard to this particular monomer, mention may be made of the book “n-Nylons, Their Synthesis, Structure and Properties”—1997 publisher J. Wiley and Sons, chapter 2.9 (pages 381 to 389) of which is devoted to Nylon 9 (or 9-Nylon). This article synthesizes the productions and studies carried out on the subject. Mentioned therein (page 384) is a process developed in Japan using oleic acid from soybean oil as raw material and consisting in carrying out an ozonolysis of the oleic acid, followed by reductive ammoniation, thus resulting in 9-aminononanoic acid.

To complete the prior art which comes from the scientific literature, it is necessary to mention the numerous articles published by E. H. Pryde and various coauthors between 1962 and 1975 in—Journal of the American Oil Chemists Society—“Amines from Aldehydic derived from the Ozonization of Soybean Esters” vol. 42, pages 824-827, which makes reference (page 824) to prior studies carried out by H. Otsuki and H. Funahashi relating to the ozonolysis of fatty acids and “Nylon-9 from Unsaturated Fatty Derivatives: Preparation and Characterization”, vol. 52, pages 473-477, in which a comparison is made between various routes of synthesis of 9-aminononanoic acid (pages 474 and 475), one of which is the nitrile route, with formation of the oleonitrile, which is subsequently subjected to oxidative ozonolysis.

With regard to the patent literature, mention may be made of patent GB 741 739 which describes the synthesis of 9-aminononanoic acid from unsaturated fatty acids of formula R—CH═CH—(CH₂)₇—COOH with a first ammoniation step resulting in the corresponding nitrile, which is subjected in a second step to oxidative ozonolysis resulting in the heminitrile of azelaic acid, which is converted in a third step by hydrogenation to 9-aminononanoic acid.

The applicant has recently filed a patent application published under No. FR 2 938 533 describing a process for synthesizing ω-amino fatty acids from fatty acids/esters of formula R₁—CH═CH—(CH₂)ₚ—COOR₂ in which R₁ is H or a hydrocarbon-based radical comprising from 4 to 11 carbon atoms and, where appropriate, a hydroxyl function, R₂ is H or an alkyl radical comprising from 1 to 4 carbon atoms and p is a whole index between 2 and 11. This process comprises two variants which both pass through the formation of an intermediate ω-unsaturated nitrile, one of the variants comprising an ammoniation step and a step of oxidative ozonolysis (of the ω-unsaturated nitrile).

Moreover, a certain number of documents published since 1990 relate to the synthesis of diacids (or diesters) from unsaturated fatty acids (or esters).

Patent application WO 93/12064 describes a process for synthesizing fatty diacids (or esters) from unsaturated fatty acids (or esters). This process uses aqueous hydrogen peroxide as oxidizing agent for the oxidative cleavage of the double bond. It is carried out in one step and in the presence of a phase-transfer agent.

European patent EP 0 666 838 describes a process for synthesizing fatty diacids (or esters) from unsaturated fatty acids (or esters). This process is carried out in two steps. The first step uses aqueous hydrogen peroxide to oxidize the double bond while forming a vicinal diol. The second step uses oxygen as oxidizing agent for obtaining the cleavage of the bond between the two carbon atoms bearing the OH functions.

This reaction to cleave the double bond of unsaturated fatty acids has also been the subject of a university study by E. Santacesaria et al. “Oxidative Cleavage of the Double Bond of Monoenic Fatty Chains in Two Steps: A New Promising Route to Azelaic and Other Industrial Products” published in Ind. Eng. Chem. Res. 2000, 39, 2766-2771, which analyzes the two oxidative cleavage steps mentioned in European patent EP 0 666 838.

The point in common between virtually all the various schemes described is a step of cleavage of the double bond of the unsaturated fatty acid by means of a strong oxidizing agent, so as to go from a long-chain unsaturated fatty acid to two reduced-chain saturated fatty molecules, one being α-ω-bifunctional and the other monofunctional.

The double-bond oxidative cleavage reaction which results in the formation of the acid function on the two carbons of the double bond is also in itself known. It can be carried out using a wide range of strong oxidizing agents.

It can, for example, be carried out by means of a strong oxidizing agent such as KMnO₄ in concentrated form and with heat. The oxidative cleavage can also be obtained via a sulfochromic route or by using ammonium chlorochromate as oxidizing agent. Angew. Chem. Int. Ed. 2000, 39, pp. 2206-2224 describes the oxidative cleavage of the double bond, either with a peracid combined with a ruthenium-based catalyst, or with H₂O₂ combined with Mo-, W- or Re-based catalysts.

However, the route which has most generally been used for decades is ozonolysis. The latter can be carried out with methyl oleate by way of example, in reductive form according to the following reaction:

H₃C—(CH₂)₇—CH═CH—(CH₂)₇—COOCH₃+(O₃,H₂)→HOC—(CH₂)₇—COOCH₃+H₃C—(CH₂)₇—COH

or in oxidative form according to the following reaction:

H₃C—(CH₂)₇—CH═CH—(CH₂)₇—COOCH₃+(O₃+O₂+H₂O)→H₃C—(CH₂)₇—COOH+COOH—(CH₂)₇—COOCH₃

The function introduced will be of the aldehyde type if the cleavage is carried out under reducing conditions and of the acid type if the cleavage is carried out under oxidizing conditions.

The choice between the two ozonolysis variants is essentially linked to the final product envisioned. The reaction processes with the formation of an ozonide and the operating conditions of these reactions have been widely described in the literature, in particular in the abovementioned articles.

The use of H₂O₂ as cleavage agent, already mentioned in patent GB 743 491, has been the subject of relatively recent studies which were mentioned above: WO 93/12064, EP 0 666 838, E. Santacesaria et al, WO 07/039,481.

The problem is that of finding a process for synthesizing the saturated or unsaturated heminitrile which is more efficient and/or less expensive than the prior processes. As it happens, the applicant has discovered that the use of H₂O₂ as oxidative cleavage agent in at least one of the steps of the process, combined with the use of an unsaturated nitrile as reagent, makes it possible to achieve performance levels which are much higher than those obtained with the prior art processes.

The subject of the present invention is therefore a process for synthesizing a heminitrile of formula CN—(CH₂)ₙ—COOH or of formula CN—R′—COOH, in which formulae n is between 4 and 13 (limits included) and R′ represents an alkylene radical comprising from 4 to 13 carbon atoms and from 0 to 2 (limits included) double bonds, with said synthesis being carried out using a compound of unsaturated fatty acid (including ester or glyceride) type of natural origin, corresponding to the formula

(R₁—CH═CH—[(CH₂)_{q}—CH═CH]ₘ—(CH₂)ᵣ—COO—)ₚ-G

in which formula:

R1 is H, or an alkyl radical having from 1 to 11 carbon atoms comprising, where appropriate, a hydroxyl function,

q is an index 0 or 1,

m and p are whole indices, m being 0, 1 or 2 and p being an integer between 1 and 3 (limits included),

if p is 1, in this case, G is an H, an alkyl radical having from 1 to 11, preferably from 2 to 11, carbon atoms, or a radical comprising two or three carbon atoms, bearing one or two hydroxyl function(s),

if p is 2, in this case, G is the residue of a diol or of glycerol bearing a hydroxyl function,

if p is 2, in this case, G is the residue of a diol or glycerol also bearing a hydroxyl function,

if p is 3, in this case, G is the residue of glycerol,

r is a whole index between 4 and 13 (limits included),

with it being possible for the C═C double bonds in said formula to be in cis or trans conformation
and
with said process comprising a first step of ammoniation of the compound of unsaturated fatty acid, ester or glyceride type, resulting in the corresponding unsaturated nitrile, which nitrile is subjected, in a second step, to an oxidative cleavage in two successive phases with the formation of intermediate compounds of vicinal diol type, using H₂O₂ as oxidizing agent, in at least one of the two phases, so as to result in said heminitrile.

Insofar as said compound of unsaturated fatty acid (including ester or glyceride) type is of natural origin, it may contain small amounts of other compounds, in particular of saturated fatty acid type of natural origin, as is the case in an oil of natural origin. Consequently, the definition of the compound of unsaturated fatty acid (including ester or glyceride) type also means a product comprising said compound of unsaturated fatty acid (including ester or glyceride) type of natural origin having the formula specified above. For example, in the case of the synthesis, according to the present invention, of the corresponding heminitrile (8-cyanooctanoic acid) from oleic oil as raw material of natural origin, this oil, depending on its purity, can comprise, inter alia and in addition, to the purely oleic major esters (including triglycerides), mixed minor esters of oleic acid and of stearic (saturated acid corresponding to oleic) and palmitic (saturated acid comprising 16 carbon atoms) acid. The limitation to the compound as defined above (according to formula) as raw material is a particular case of the invention.

With regard to the terminology “between a and b” used above and for the rest of the description of the invention, it generally means, unless otherwise indicated, the inclusion of the limits a and b and is to be considered equivalent to the expression “ranging from a to b”, which expression may also be used.

The term “ester” means simple ester, the “glyceride” (mono-, di- or tri) being considered to be a complex ester.

The heminitrile of formula CN—(CH₂)ₙ—COOH can be obtained from a compound of unsaturated fatty acid type corresponding to the formula R₁—CH═CH—(CH₂)ᵣ—COOG, in which formula G is an H, an alkyl radical having from 1 to 11 and preferably from 2 to 11 carbon atoms, or a radical comprising two or three carbon atoms, bearing one or two hydroxyl function(s).

The heminitrile of formula CN—R′—COOH can be obtained from a compound of unsaturated fatty acid type corresponding to the formula (R₁—CH═CH—[(CH₂)_{q}—CH═CH]ₘ—(CH₂)ᵣ—COO—)ₚ-G, with R₁, G, m, p, q and r being defined as above.

According to one particular case of the process described above, for p=1, G can be a methyl, which would correspond, in the case where said unsaturated fatty acid is oleic acid, to a methyl oleate ester.

Said second step of oxidative cleavage can, where appropriate, be preceded by an ethenolysis of said nitrile if it is desired to use an ω-unsaturated nitrile as substrate of the cleavage reaction. This use is of more particular advantage in the preparation of ω-amino acids as polyamide monomers. In this case, the process according to the invention may comprise an intermediate step of ethenolysis (or of cross metathesis with a light olefin) of the nitrile resulting from said ammoniation step, so as to result in an ω-unsaturated nitrile, this being before the second step where said ω-unsaturated nitrile is subjected to said oxidative cleavage.

More particularly, this ethenolysis (or metathesis with a light olefin) can be applied to the oleonitrile resulting from the ammoniation of an oleic acid compound, such as oleic acid or ester or the corresponding glyceride. More precisely, the oleonitrile as obtained in the first step can be used in the preparation of 9-amino nonanoic acid (monomer of polyamide 9), either via the route comprising the prior ethenolysis of the oleonitrile, or via the route of oxidative cleavage directly on the oleonitrile, the latter route being simpler (without ethenolysis or metathesis).

Since the ethenolysis of the nitrile of said fatty acid is in fact a metathesis reaction of said nitrile in the presence of ethylene, it is also possible to carry out this metathesis in the presence of propylene, of 1-butene or of 2-butene. Preferably, this intermediate metathesis is carried out in the presence of ethylene or of 1-butene, and more preferentially in the presence of ethylene (ethenolysis).

According to a more preferred embodiment, said process does not comprise any intermediate step of ethenolysis or of metathesis in general, said process consequently being simpler.

It is known, see in particular the abovementioned publication by E. Santacesaria, that the reaction for oxidative cleavage of a double bond is carried out in two phases. During the first phase, the double bond is oxidized, which results in the formation of a vicinal diol. In the second phase, the carbon-carbon bond between the two hydroxyl functions is broken, with the formation of, on the one hand, a saturated or unsaturated acid and, on the other hand, a heminitrile, it being possible for the unsaturated acid to be obtained in particular in the case where the feedstock comprises polyunsaturated fatty acids. More precisely, in the latter case of polyunsaturated fatty acids, depending on the position of the double bond subjected to the oxidative cleavage, it is possible to obtain, as product, either an unsaturated acid or an unsaturated heminitrile and therefore, more probably, a mixture of the two. The reaction scheme with oleonitrile by way of example is the following:

CH₃—(CH₂)₇—CH═CH—(CH₂)₇—CN+oxidizing agent→CH₃—(CH₂)₇—CHOH—CHOH—(CH₂)₇—CN

CH₃—(CH₂)₇—CHOH—CHOH—(CH₂)₇—CN+oxidizing agent→CH₃—(CH₂)₇—COOH+COOH—(CH₂)₇—CN

In the case of linoleonitrile, the reaction scheme is:

either

CH₃—(CH₂)₄—CH═CH—CH₂—CH═CH—(CH₂)₇—CN+oxidizing agent→

CH₃—(CH₂)₄—CHOH—CHOH—CH₂—CH═CH—(CH₂)₇—CN

and

CH₃—(CH₂)₄—CHOH—CHOH—CH₂—CH═CH—(CH₂)₇—CN+oxidizing agent→CH₃—(CH₂)₄—COOH+HOOC—CH₂—CH═CH—(CH₂)₇—CN

or

CH₃—(CH₂)₄—CH═CH—CH₂—CH═CH—(CH₂)₇—CN+oxidizing agent→CH₃—(CH₂)₄—CH═CH—CH₂—CHOH—CHOH—(CH₂)₇—CN

and

CH₃—(CH₂)₄—CH═CH—CH₂—CHOH—CHOH—(CH₂)₇—CN+oxidizing agent→CH₃—(CH₂)₄—CH═CH—CH₂—COOH+HOOC—CH₂)₇—CN

And the final product is a mixture of the two heminitriles. When the feedstock comprises polyunsaturated acids, i.e. when m is not zero (when m is equal to 1 or 2) and one of the heminitriles corresponds to the general formula mentioned above and with n=r+(q+2)*m, in this case, a hydrogenation of C═C double bond(s) must be carried out during a subsequent or prior step. This hydrogenation may be carried out in particular after the formation of a vicinal diol so as to force the oxidative cleavage to take place in a single position or at the same time as the hydrogenation of the nitrile function to an amine function.

In this variant of the process, with polyunsaturated nitriles, the first oxidation phase can also result in the partial formation of two vicinal diols, which can in the end lead to the formation of by-products such as short diacids.

According to varied embodiments of the invention, the following cases are possible:
the heminitrile of formula CN—(CH₂)ₙ—COOH is obtained from a compound of unsaturated fatty acid type corresponding to the formula R₁—CH═CH—(CH₂)ᵣ—COOG, in which G is an H, an alkyl radical having from 1 to 11 and preferably from 2 to 11 carbon atoms, or a radical comprising two or three carbon atoms and bearing one or two hydroxyl function(s);
the heminitrile of formula CN—R′—COOH is obtained from a compound of unsaturated fatty acid type corresponding to the formula (R₁—CH═CH—[(CH₂)_{q}—CH═CH]ₘ—(CH₂)ᵣ—COO—)ₚ-G, with G, R1, m, p, q and r as defined above.

The process of the invention can be carried out according to several other variants.

According to a first variant, the first phase of the second step is carried out using H₂O₂ as oxidizing agent in the presence of a catalyst and the second phase of oxidative cleavage is carried out by oxidation with pure or diluted oxygen and/or with air as oxidizing agent (the oxidizing agent being molecular oxygen O₂ in both cases), optionally in the presence of a second catalyst.

According to a second variant, the first phase is carried out using H₂O₂ as oxidizing agent in the presence of a catalyst and the second phase of oxidative cleavage is carried out in a second reactor by oxidation by means of H₂O₂ as oxidizing agent, optionally in the presence of another catalyst.

According to a third variant, the two phases are carried out successively in a single reaction medium and using H₂O₂ as oxidizing agent in the presence of a single catalyst. WO 93/12064 describes a process using H₂O₂ as sole oxidizing agent for synthesizing a diacid from an unsaturated fatty acid. This process requires the use of a phase-transfer agent.

According to a fourth variant, the two phases are carried out successively in a reactor comprising two zones: the first zone being fed with H₂O₂ as oxidizing agent and in the presence of a first catalyst, and the second zone being fed with O₂ (air or oxygen) as oxidizing agent and in the presence of a second catalyst, the reaction medium being moved from one zone to the other by any suitable means.

Thus, the first phase of said second step (oxidation) resulting in the vicinal diols can be carried out by oxidation of a double bond or double bonds in the presence of H₂O₂ as oxidizing agent, in the presence of an oxidation catalyst.

The expression “compound of natural unsaturated fatty acid type” is intended to mean an acid or the corresponding unsaturated fatty ester (including glyceride) derived from the plant or animal environment, including algae, more generally derived from the plant kingdom and therefore renewable. This acid compound comprises at least one olefinic unsaturation, located in position x relative to the acid group (delta x) and comprises between 7 and 24 (limits included) carbon atoms per molecule. This acid compound can be employed after hydrolysis of natural oils, but also directly in the form of glycerides.

These various acid compounds are derived from vegetable oils extracted from various oleagineous plants, such as sunflower, rape, castor oil plant, Lesquerella, Camelina, olive, soya, palm tree, Sapindaceae, in particular avocado, sea buckthorn, coriander, celery, dill, carrot, fennel, mango or Limnanthes alba (meadowfoam), from microalgae or from animal fats.

The location of the double bond makes it possible to determine the formula of the final heminitrile and the acid compound will therefore be chosen according to the heminitrile desired.

In order to obtain a heminitrile comprising 6 carbon atoms, petroselenic acid (cis-6-octadecenoic acid), its derivative 6-heptenoic acid obtained by ethenolysis and α-linolenoic acid (6,9,12-octadecatrienoic acid), which can be obtained, for example, from coriander, will be used as raw material.

In order to obtain a heminitrile comprising 8 carbon atoms, cis-8-eicosenoic acid, cis-5,8,11,14-eicosatrienoic acid (arachidonic acid) and ricinoleic acid, which give, after dehydration, conjugated 8,10-octadecadienoic acid, will be used as raw material.

In order to obtain a heminitrile comprising 9 carbon atoms, use may be made of a wide range of fatty acids, for instance caproleic (cis-9-decenoic) acid, palmitoleic (cis-9-hexadecenoic) acid, myristoleic (cis-9-tetradecenoic) acid, oleic (cis-9-octadecenoic) acid, 9-decenoic acid obtained by ethenolysis of an oleic acid, for example, elaidic (trans-9-octadecenoic) acid, and ricinoleic (12-hydroxy-cis-9-octadecenoic) acid, gadoleic (cis-9-eicosenoic) acid, linoleic (9-12-octadecadienoic) acid, rumenic (9-11-octadecadienoic) acid, conjugated linoleic (9-11-octadecadienoic) acid. These acids can be obtained from sunflower, rape, castor oil plant, olive, soya, palm tree, flax, avocado, seed buckthorn, coriander, celery, dill, carrot, fennel and Limnanthes (meadowfoam).

In order to obtain a heminitrile comprising 10 carbon atoms, use will be made of 10,12 conjugated linoleic acid (10-12-octadecadienoic acid) or 10-undecylenic acid obtained by thermal cracking of ricinoleic acid methyl ester. In order to obtain a heminitrile comprising 11 carbon atoms, use may be made of vaccenic (cis-11-octadecenoic) acid, gondoic (cis-11-eicosenoic) acid, lesquerolic (14-hydroxy-cis-11-eicosenoic) acid, and cetoleic (cis-11-docosenoic) acid which can be obtained from Lesquerella oil (lesquerolic acid), from Camelina sativa oil (gondoic acid), the oil of a plant of the family Sapindaceae, from fish fat and from microalgae oils (cetoleic acid) by dehydration of 12-hydroxystearic acid (12-HSA), itself obtained by hydration of ricinoleic acid (vaccenic acid and its trans equivalent) and of conjugated linoleic acid (9,11-octadecadienoic acid), obtained, for example, by dehydration of ricinoleic acid.

In order to obtain a heminitrile comprising 12 carbon atoms, use may be made of (cis or trans) 12-octadecenoic acid obtained, for example, by dehydration of 12-hydroxystearic acid (12-HSA), the 12-HSA being obtained, for example, by hydration of ricinoleic acid, 10,12 conjugated linoleic acid (10,12-octadecadienoic acid) or 12-tridecenoic acid obtained, for example, by thermal cracking of the ester (in particular methyl ester) of lesquerolic acid.

In order to obtain a heminitrile comprising 13 carbons, use may be made of erucic (cis-13-docosenoic) acid or brassylic (trans-13-docosenoic) acid which can, for example, be obtained from erucic rape, Honesty or Crambe maritime (sea kale), and (cis or trans) 13-eicosenoic acid obtained, for example, by dehydration of 14-hydroxyeicosanoic acid, itself obtained by hydrogenation of lesquerolic acid.

In order to obtain a heminitrile comprising 14 carbon atoms, use may be made of (cis or trans) 14-eicosenoic acid obtained, for example, by dehydration of 14-hydroxyeicosanoic acid (14-HEA), itself obtained by hydrogenation of lesquerolic acid.

In order to obtain a heminitrile comprising 15 carbon atoms, use may be made of nervonic (cis-15-tetracosoic) acid which can be obtained from Malania oleifera and from Honesty (Lunaria annua, also known as Pope's coin or money plant).

The acid compounds which are the most important in nature, in order of importance, are those which give C9-unsaturated acids (unsaturated in position 9), then C13-unsaturated acids and then C11-unsaturated acids, since they are the most widely available.

One of the acids which is preferred, with gondoic acid and lesquerolic acid, for obtaining a heminitrile comprising 11 carbon atoms, is vaccenic acid.

Preferably, the vaccenic acid is of natural origin, i.e. derived from the plant or animal environment, including algae, more generally from the plant kingdom and therefore renewable. Thus, according to one preferred embodiment, the subject of the invention is a process for synthesizing a heminitrile from vaccenic acid of natural origin, as compound of unsaturated fatty acid type (including ester or glyceride derivatives).

The pathways described below make it possible to obtain a vaccenic acid of renewable origin:
the vaccenic acid can be obtained directly from the plants, in particular by extraction, from mango pulp, from sea buckthorn, from sea buckthorn oil or from derivatives of animal origin such as butter.
The vaccenic acid can also be obtained by genetically modifying plants such as safflower, camelina or else Arabidopsis thaliana as is described by Nguyen et al., in Plant Physiology, December 2010, vol. 154, pp 1897-1904.
The vaccenic acid can, finally, be obtained from genetically modified bacteria or yeasts, for example Escherichia coli, as is described by Mendoza et al, in Journal of Bacteriology September 1982, pp 1608-1611.
A final route for obtaining vaccenic acid is dehydration or, respectively, ammoniation of 12-hydroxystearic acid.

With regard to gondoic acid (cis 11-eicosenoic acid), it can be used, like vaccenic acid and lesquerolic acid, preferably, for the preparation of undecanoic heminitrile.

Preferably, when gondoic acid is used, it is of natural origin, i.e. of plant origin (which includes algae) or of animal origin. In one preferred embodiment, the subject of the invention is a process for oxidative cleavage of an unsaturated fatty nitrile obtained from gondoic acid of natural origin, so as to obtain the corresponding heminitrile.

The following routes make it possible to obtain a gondoic acid of natural origin:
the gondoic acid (cis-11-eicosenoic) acid can be obtained directly from plants, in particular by extraction, from Camelina (Camelina sativa) oil which contains more than 15% of gondoic acid, from erucic acid-rich rapeseed oil, from Crambe, from honesty which generally contains from 2% to 15% of gondoic acid, from Alyssum maritimum (gondoic acid content of 41.8%), from Selenia grandis (gondoic acid content of 58.5%) or from Marshallia caespitosa (gondoic acid content of 43.9%).
The gondoic acid can also be obtained by genetically modifying plants such as Camelina or else Arabidopsis thaliana.
The gondoic acid can be obtained from genetically modified bacteria or yeasts for example Escherichia coli.
A final route for obtaining gondoic acid is hydrolysis of jojoba oil, which is in fact a vegetable wax (called oil because the wax is liquid at ambient temperature). This vegetable wax (fatty ester) contains approximately 35% by weight of gondoic acid. The hydrolysis thereof gives a mixture of fatty acids containing gondoic acid (approximately 70% by weight of the fatty acids) and long-chain fatty alcohols which are separated.

JP9-278706 and JP9-279179 describe processes for obtaining gondoic acid of high purity from the hydrolysis of jojoba oil.

More particularly, the gondoic acid can be obtained via the following routes:
1) as described in the cited patents JP9-278706 and JP9-279179, from jojoba oil. After hydrolysis of the oil and a first distillation, extraction is carried out with urea and an acid fraction is obtained which is rich in gondoic acid and also contains a few traces of erucic acid, preferably without any other unsaturated acid.
2) From HEAR (High Erucic Acid Rapeseed) erucic rapeseed oil containing, for example, from 5% to 15% of gondoic acid. Crambe oil and Honesty oil can also, for example, be used.
3) From a rapeseed variety selected for its higher gondoic acid content, for example harvested before total maturity of the plant, since the fatty-chain elongation mechanism involves the intermediate production of higher concentrations of gondoic acid in the plant.
4) From Camelina oil, rich in gondoic acid.
5) From other plants such as Alyssum maritimum rich in gondoic acid (gondoic acid content of 41.8%), Selenia grandis (content of 58.5%), or Marshallia caespitosa (content of 43.9%).

All the plants mentioned above and others in genetically modified varieties can be used for providing gondoic acid-enriched oils.

For all the fatty acids mentioned above, both acids having a cis conformation and acids having a trans conformation may be used.

Among the preferred acids (and derivatives) that can be used as raw materials for the synthesis of the heminitriles of the invention, mention may be made, for the C9 heminitriles, of oleic acid (or ester or glyceride derivative) and, for the C11 heminitriles, of lesquerolic acid, vaccenic acid and gondoic acid, according to the process of the invention involving the oxidative cleavage of the corresponding unsaturated nitrile.

Thus, the oxidative cleavage of oleonitrile results in the heminitrile of nonanedioic acid (azelaic acid) which can be used in the preparation of 9-aminononanoic acid, a monomer of polyamide 9 (Nylon 9), by hydrogenation of its nitrile function and conversion thereof to an amine function.

The oxidative cleavage of the nitriles of lesquerolic acid, of vaccenic acid and of gondoic acid results, in the three cases, in the heminitrile of undecanedioic acid, which, via the hydrogenation of its nitrile function and conversion thereof into an amine function, can be used in the preparation of 11-aminoundecanoic acid, a monomer of polyamide 11 (Nylon 11).

Preferably, the process of the invention uses oleic acid, lesquerolic acid, vaccenic acid or gondoic acid of natural origin (renewable source), as unsaturated fatty acid (or ester or glyceride derivative) used as starting raw material.

Thus, according to the process of the invention, the preferred nitrile used in the second step is oleonitrile, the nitrile of lesquerolic acid, the nitrile of vaccenic acid or the nitrile of gondoic acid.

According to one particular case, said unsaturated fatty acid of natural origin involved in the process according to the invention is oleic acid or a corresponding ester or glyceride.

According to another particular case, said unsaturated acid of natural origin is gondoic acid (cis-11-eicosenoic acid) or a corresponding ester or glyceride.

Thus, the ammoniation of oleic acid (or ester or glyceride) results, according to this process, via oxidative cleavage, in the C9 heminitrile, which can be used to prepare the C9 amino acid (9-aminononanoic acid) by adding an additional step of hydrogenation (of the nitrile function) to said process of the invention.

In the same way, lesquerolic acid (14-hydroxy-11-eicosenoic acid), vaccenic acid (cis 11-octadecenoic acid) and gondoic acid (cis-11-eicosenoic acid) can result in the preparation of the C11 amino acid (11-aminoundecanoic acid), always involving the oxidative cleavage of the unsaturated nitrile corresponding to said unsaturated fatty acids, which cleavage is followed by the hydrogenation of the corresponding heminitrile. According to this process, for the C11 heminitrile, vaccenic acid and gondoic acid are preferred, and vaccenic acid is even more preferred.

The synthesis of nitriles from acids using ammonia is well known to those skilled in the art. In this respect, reference may be made to the Kirk-Othmer encyclopedia and to patent GB 741 739 already mentioned. The reaction scheme can be summarized in the following way:

R—COOH+NH₃→[R—COO⁻NH₄⁺]→[R—CONH₂]+H₂O→RCN+H₂O

This scheme applies just as much to natural fatty acids (esters) as to ω-unsaturated fatty acids.

The process can be carried out batchwise in the liquid or gas phase or continuously in the gas phase. The reaction is carried out at high temperature and above 250° C. and in the presence of a catalyst which is generally a metal oxide, and most commonly zinc oxide. The continuous elimination of the water formed, while additionally carrying over the unreacted ammonia, enables rapid completion of the reaction. Liquid-phase ammoniation is very suitable for long fatty chains (comprising at least 10 carbon atoms). However, when operating with shorter chain lengths, gas-phase ammoniation may become more suitable.

It is also known practice to carry out the ammoniation using urea or cyanuric acid as ammoniation agent (see GB 641 955 already mentioned).

The difficult step is the cleavage. Indeed, the choice of the oxidizing agent and of the fatty acid derivative subjected to this operation are essential for obtaining good results. The choice of oxidizing agent falls on H₂O₂. It is an inexpensive “green” oxidizing agent. It has many advantages over ozone (O₃), oxygen (O₂) and the other strong oxidizing agents, such as permanganates, periodates and other strong oxidizing agents. It is easy to handle, nontoxic, and available in large amounts in liquid form. It is easier to use in the reaction since it enables a moderate reaction temperature to be used, compared with O₃, which requires cold. In addition, it is possible to operate at a pressure close to atmospheric pressure, whereas O₂ requires working under pressure. Its oxidation reaction, compared with those using O₂ or O₃, exhibits a lower exothermicity and allows good dissolution of the oxidizing agent in the medium. In addition, it needs only water as by-product, avoiding the presence of residues that are difficult to treat (toxic osmium or periodate giving a halogenated compound or permanganate or other strong oxidizing agent).

The amount of H₂O₂ introduced into the reaction medium is an important factor. This amount is always at least equal to the stoichiometry of the reaction under consideration (i.e. at least the stoichiometric amount-of H₂O₂). The first phase of the (second) step of oxidative cleavage, resulting in the formation of the vicinal diol, has a stoichiometry of 1 (1/1). The amount of H₂O₂ injected is such that the H₂O₂/unsaturated nitrile molar ratio is generally between 1/1 and 4/1 (limits included). Thus, for the first phase of the oxidative cleavage step, H₂O₂ can be injected into the medium in an amount representing from 1 to 4 molar equivalents of the unsaturated nitrile to be oxidized, i.e. an H₂O₂/unsaturated nitrile molar ratio ranging from 1 to 4, more particularly in the form of an aqueous solution having an H₂O₂ content of between 30% and 70% (limits included) by weight, preferably between 50% and 70% (limits included) by weight and more preferably between 60% and 70% (limits included) by weight, and preferably in the presence of a catalyst consisting of tungsten derivatives, molybdenum derivatives or vanadium derivatives, and more particularly chosen from tungstic acid (H₂WO₄), the sodium salt of this acid (Na₂WO₄) combined with H₃PO₄, molybdic acid (H₂MoO₄) and its sodium salt (Na₂MoO₄), heteropoly acids such as H₃[PMo₁₂O₄₀], H₄[SiMo₁₂O₄₀], H₄[SiW₁₂O₄₀], H₃[PW₁₂O₄₀] or (NH₄)₁₀[H₂W₁₂O₄₂], sodium metavanadate (Na₃VO₄), ammonium metavanadate ((NH₄)₃VO₄), and their alkali metal salts.

When the second phase of the oxidative cleavage step also uses (like the first phase) H₂O₂ as oxidizing agent, according to the second and the third variant of the process of the invention, in this case, the reaction for cleavage of the vicinal diol formed has a stoichiometry of 3 (3/1). The amount of H₂O₂ injected will then be such that the H₂O₂/vicinal diol molar ratio is between 3/1 and 10/1 (limits included). Thus, the second phase of oxidative cleavage of the vicinal diols can be carried out with H₂O₂ as agent for cleavage of the C—C bond between the vicinal hydroxyls, injected in the form of an aqueous solution having an H₂O₂ content of between 30% and 70% (limits included) by weight (or by mass), preferably between 50% and 70% (limits included) by weight and more preferably between 60% and 70% (limits included) by weight, and such that the H₂O₂/vicinal diol molar ratio is between 3/1 and 10/1 (limits included).

For the third variant of the process according to the present invention, wherein the reaction is carried out in one and the same reactor (one pot reaction), the H₂O₂/unsaturated nitrile molar ratio is between 4/1 and 15/1 (limits included).

The aqueous hydrogen peroxide is introduced in the form of an aqueous solution. The concentration of this solution is also to be taken into consideration, and it is between 30% and 70% (limits included) by weight (by mass), preferably between 50% and 70% (limits included) by weight and more preferably between 60% and 70% (limits included) by weight.

According to one advantageous implementation variant of the process of the invention, for a phase using H₂O₂ as oxidizing agent, the catalyst will be introduced sequentially at the time the corresponding reaction phase is carried out. Instead of introducing all of the catalysts into the reaction medium at the beginning of the reaction, said catalyst will be introduced in small amounts throughout the process, H₂O₂ for its part being introduced continuously. In one particularly advantageous variant, H₂O₂ and the catalyst are introduced sequentially. It may also be envisioned to continuously introduce the catalyst at a very low dose, like H₂O₂, taking care, however, to avoid any prior contact between them. It is therefore possible to carry out a sequential injection of the catalysts through the course of the reaction process.

When the process is carried out with O₂ as oxidizing agent for cleaving the C—C bond between the two vicinal hydroxyls in the second phase of the second step of the process of the invention, in this case, the amount of O₂ introduced will be at least equal to the stoichiometry (stoichiometric amount) required for the reaction, and preferably with an O₂/vicinal diol molar ratio of between 3/2 and 100/1 (limits included). In practice, it is possible to work with a very large excess of air, for example by bubbling air into the medium, since it is not necessary for all the oxygen to react, and thus to work at a low temperature in order to have better control of the selectivity.

The catalysts that can be used for these two oxidative cleavage reactions are generally known to those skilled in the art.

The catalysts of the first phase preferably consist of tungsten derivatives, molybdenum derivatives or vanadium derivatives. By way of example, mention may be made of tungstic acid (H₂WO₄), the sodium salt of this acid (Na₂WO₄) combined with H₃PO₄, molybdic acid (H₂MoO₄) and its sodium salt (Na₂MoO₄), heteropoly acids such as H₃[PMo₁₂O₄₀], H₄[SiMo₁₂O₄₀], H₄[SiW₁₂O₄₀], H₃[PW₁₂O₄₀] or (NH₄)₁₀[H₂W₁₂O₄₂], sodium metavanadate (Na₃VO₄) or ammonium metavanadate ((NH₄)₃VO₄). Generally, the alkali metal salts of the acids mentioned above are also suitable.

Catalysts of this type will be used in the variant of the process carried out in a single reactor with H₂O₂ as oxidizing agent.

The amount of catalysts that are used in this first phase are generally between 0.03% and 2% (limits included) by weight relative to the weight of nitrile treated, and preferably between 0.5% and 2% (limits included) by weight.

During the second phase of cleavage of the vicinal diol using molecular oxygen as oxidizing agent, it is possible to use catalysts based on cobalt, in the form of cobalt acetate, such as Co(Ac)₂.4H₂O, chloride or sulfate, or salts of Cu, Cr, Fe or Mn, and also some of the catalysts used during the first phase such as tungstic acid (H₂WO₄) and its sodium salt Na₂WO₄ and mixtures of the metals as mentioned above, in particular Co/W.

Thus, according to one particular embodiment of the process of the invention, the second phase of said second step of oxidative cleavage of the vicinal diols can be carried out with O₂ as oxidizing agent for cleavage of the C—C bond between said vicinal hydroxyls, more particularly in the presence of a catalyst chosen from cobalt salts such as cobalt acetate (Co(Ac)₂.4H₂O), chloride and sulfate or salts of Cu, Cr, Fe or Mn, and also the catalysts used during the first phase, chosen from tungstic acid (H₂WO₄) and its sodium salt Na₂WO₄ and Co/W mixtures.

Even more particularly, in the second phase of the second step of the process of the invention, the reaction can be carried out with O₂ as oxidizing agent for cleavage of the C—C bond between the two vicinal hydroxyls and, in this case, the amount of O₂ introduced will be at least equal to the stoichiometry (stoichiometric amount) required for said reaction, and preferably with an O₂/vicinal diol molar ratio of between 3/2 and 100/1 (limits included), and even more preferably with the reaction being carried out at a temperature of between 20 and 80° C. (limits included) and preferably between 40 and 70° C. (limits included) and even more particularly at a pressure of between 1 and 50 bar (limits included), preferably between 1 and 20 bar (limits included) and more preferentially between 5 and 20 bar (limits included).

According to another particular variant of the process of the invention, said second phase of oxidative cleavage of the vicinal diols is carried out with H₂O₂ as oxidizing agent for cleaving the C—C bond between the vicinal hydroxyls and preferably with said H₂O₂ being injected in the form of an aqueous solution having an H₂O₂ content of between 30% and 70% (limits included) by weight (by mass), preferably between 50% and 70% (limits included) by weight and more preferably between 60% and 70% (limits included) by weight and with an H₂O₂/vicinal diol molar ratio of between 3/1 and 10/1 (limits included).

The amounts of catalysts used in this second phase are between 0.1 mol % and 3 mol % (limits included) relative to the diol treated, and preferably between 1 mol % and 2 mol % (limits included).

More particularly, according to the process of the invention, the catalysts are injected sequentially through the course of the reaction process.

The second step of the process of the invention is carried out at a temperature of between 20 and 80° C. (limits included) and preferably between 40 and 70° C. (limits included). The reaction can be carried out in a wide pressure range, at a pressure of between 1 and 50 bar (limits included), preferably between 1 and 20 bar (limits included) and more preferably at a pressure approximately equal to atmospheric pressure or slightly above atmospheric pressure and between 1 and 5 bar (limits excluded). However, when the second phase of the oxidative cleavage is carried out with molecular oxygen (O₂), it is possible to use a pressure which is higher than the pressure used during the first phase, and generally between 5 and 20 bar (limits included).

According to one implementation variant of the process of the invention, two separate reactors are used for implementing the second step, with one reactor for the first phase and another for the second phase. In this case, it is advantageous to have recycling into the first-phase reactor of the diols formed at the end of this phase. The degree of recycling is generally between 1% and 10% by weight (limits included) relative to the starting nitrile entering the reaction. Thus, in this case, two separate reactors are used for implementing the second step, and the effluent resulting from the first phase (1^{st} reactor) is subjected to a partial separation of the aqueous and organic fractions, thus enabling the partial elimination of the aqueous fraction and the recycling at the top of the first-phase (1^{st}) reactor of a part of the organic fraction, representing from 1% to 10% by weight of said unsaturated nitrile.

According to another variant of the process of the invention, use is made of a single reactor with H₂O₂ as sole oxidizing agent for the two phases of the second step and with the H₂O₂/nitrile molar ratio being between 4/1 and 10/1 (limits included).

The heminitrile obtained at the end of the process can be used as a substrate or raw material for synthesizing ω-amino acids. The heminitrile is subjected to a reaction for reduction with hydrogen of the nitrile function according to the following reaction scheme in the case of the oleonitrile derivative:

HOOC—(CH₂)₇—CN→2H₂→HOOC—(CH₂)₇—CH₂NH₂

This reduction of the nitrile function to a primary amine and the obtaining of ω-amino fatty acids (esters), from heminitriles in the case in point, is well known to those skilled in the art. The reduction step consists of a conventional hydrogenation. Numerous catalysts can be used, but Raney nickel and Raney cobalt will preferentially be used. In order to promote the formation of the primary amine, the process is carried out with a partial ammonia pressure.

The heminitrile obtained at the end of the process can be used as a synthesis substrate for synthesizing dinitrile by a subsequent reaction with ammonia, according to the following reaction scheme:

CN—(CH₂)ₙ—COOH+NH₃→CN—(CH₂)ₙ—CN+2H₂O

This ammoniation of the acid function to a nitrile is well known to those skilled in the art, see, in this respect, GB 741 739 already mentioned. The reaction is carried out at high temperature, preferably above 250° C., and in the presence of a catalyst which is generally a metal oxide and most commonly zinc oxide.

The hydrogenated dinitrile results in a diamine which can be used in numerous applications, including the preparation of polyamides, in particular in combination with diacids.

The heminitrile obtained at the end of the process can be used as a substrate for synthesizing diacid by hydrolysis of the nitrile function according to the following reaction scheme:

CN—(CH₂)ₙ—COOH+2H₂O→HOOC—(CH₂)ₙ—COOH+NH₃

The hydrolysis is generally carried out under acid conditions.

The diacid can be used in numerous applications, including the preparation of polyamides, in particular in combination with diamines.

In the first variant of the process, the first phase of oxidation of the nitrile is carried out in a first reactor operating at a temperature of between 20 and 70° C. (limits included) and a pressure of between 1 and 5 bar (limits included), in the presence of a catalyst consisting of tungstic acid or an equivalent catalyst, with an amount of H₂O₂ of between 1 and 2 molar equivalents (i.e. from 1 to 2 mol of H₂O₂ per mole of compound treated), introduced in the form of an aqueous solution of H₂O₂ with a content of between 35% and 70% by weight (limits included). At the end of this first phase, where appropriate, after extraction of the water, the reaction medium is transferred into a second reactor where it is subjected to oxidation with O₂ in the presence of a cobalt-based catalyst or an equivalent catalyst, at a temperature generally of between 40 and 70° C. (limits included) and at a pressure of between 5 and 20 bar (limits included), with an excess of molecular oxygen.

When two separate reactors are used for implementing the second step, the effluent resulting from the first phase can be subjected to a partial separation of the aqueous and organic fractions, thus enabling the partial elimination of the aqueous fraction and the recycling at the top of the first-phase reactor of a part of the organic fraction representing from 1% to 10% by weight of the unsaturated nitrile.

In the second variant, the first phase is carried out as in the first variant and the second cleavage phase is carried out in a second reactor by oxidation by means of H₂O₂ in the presence of tungstic acid or of another catalyst. At the end of the first phase, the reaction medium can be subjected to an extraction of the aqueous phase and also to a partial withdrawal of the organic phase for recycling into the first-phase reactor. The temperature and pressure conditions during the second phase are generally “milder” than in the first variant.

In the third variant, the two phases are carried out successively in a single reactor, the reaction medium being oxidized with H₂O₂ in the presence of a single catalyst generally consisting of tungstic acid or an equivalent catalyst. The amount of H₂O₂ introduced is between 4 and 10 (limits included) molar equivalents (i.e. from 4 to 10 mol of H₂O₂ per mole of nitrile) in the form of a solution with an H₂O₂ content of between 35% and 70% by weight (limits included). Thus, when a single reactor is used for the two phases with H₂O₂ as sole oxidizing agent, the H₂O₂/nitrile molar ratio can be between 4/1 and 10/1 (limits included).

In the fourth process variant, the two phases are carried out successively in a reactor comprising two zones, the first being fed with H₂O₂ as oxidizing agent in the presence of a first catalyst and the second with O₂ (air) in the presence of a second catalyst, said reactor being provided with means for moving the reaction medium from the first-phase zone to the second-phase zone. The moving of the reaction medium from one zone to the other can be carried out, for example, in a rotating reactor of centrifuge type, a cone- or disk-shaped reactor, or any other device for producing thin layers (thin films) of liquid. By way of example in this respect, mention may be made of a Spinning Disk Reactor as described in the technical documentation of the company Protensive (Protensive Limited, BioScience Centre, International Centre for Life, Times Square, Newcastle upon Tyne, NE1 4EP, United Kingdom), a Rotating Packed Bed Reactor (Dow Chemical Company) or else reactors such as those sold by the company Myers Vacuum, Inc. which are in the form of tanks which are kept rotating. By way of example of equipment producing thin layers of liquid, mention may be made of those described in Techniques de I'Ingénieur [Techniques of the Engineer], number J2360-9 from 1988. In all these technologies, the force of gravity is replaced with centrifugal force or with a mechanical force in order to maintain a thin film of liquid and to thus promote gas-liquid matter transfer. This type of technology is particularly suitable for the present process since the reaction media not found to be sensitive (the reaction is sensitive) not only to temperature variation, but also to the viscosity of the medium (viscous media).

In the devices of centrifuge type, the first reaction phase is carried out in proximity to the axis of rotation fed with substrate (fatty nitrile), catalyst (tungstic acid) and H₂O₂ in an amount representing between 1 and 4 (limits included) mole of H₂O₂ per mole of nitrile (molar equivalents), in aqueous solution at a content of between 35% and 70% by weight (including limits), and the second phase is carried out in proximity to the periphery of the device where excess O₂ is injected, the second-phase catalyst being, for its part, always introduced into a central zone of the device. The final product of the reaction is recovered by overflowing at the periphery. The reactors which may be suitable all provide a thin thickness of liquid film, swept in countercurrent or concurrent mode with a gas stream containing molecular oxygen.

The use of this type of device may also be advantageous in the variant of the process wherein the oxidation phase is carried out with molecular oxygen in a reactor independent of the first-phase reactor.

According to one more particular embodiment of the process as described above according to the invention, said unsaturated fatty acid, used in the first step to prepare said unsaturated nitrile of fatty acid, is prepared in a prior step of said process, comprising the hydrogenation of a (suitable) corresponding starting hydroxylated unsaturated fatty acid so as to obtain the corresponding hydrogenated acid, said hydrogenation being followed by dehydration of said hydrogenated acid. More particularly, this process applies to vaccenic acid as unsaturated fatty acid, with the corresponding starting hydroxylated unsaturated fatty acid being ricinoleic acid and the corresponding hydrogenated acid being 12-hydroxystearic acid (12-HSA).

The process of the invention, in addition to the manufacture of said heminitrile, can be used directly or indirectly for preparing an ω-amino acid equivalent (corresponding) to said heminitrile, with said process comprising an additional step of hydrogenation of the nitrile function of said heminitrile by converting it into a corresponding amine function.

According to this use, the process of the invention can result in the preparation of 9-amino nonanoic acid from oleonitrile or in 11-aminoundecanoic acid from the nitriles of lesquerolic acid or of vaccenic acid or of gondoic acid, preferably from the nitriles of vaccenic acid or of gondoic acid and more preferentially from the nitriles of vaccenic acid.

The process of the invention can also be used for preparing the diamines and/or diacids corresponding to said heminitrile of the invention, thus obtained by means of said method, as already described above. Thus, said diamines and/or diacids can be used, like said ω-amino acids, as monomers for obtaining polyamides from raw materials which are of natural origin and from a renewable source.

The preferred use of the process for preparing a heminitrile according to the present invention relates to the preparation of polyamide monomers selected from ω-amino acids and/or the diamines and/or the diacids equivalent to said heminitrile and/or also relates to the production of polyamides, by polymerization of said monomers.

More particularly, said use relates to the preparation of a monomer which is an ω-amino acid equivalent to said heminitrile and said process comprising an additional step of hydrogenation of the nitrile function of said heminitrile and the conversion thereof into the corresponding amine function.

Even more specifically, this use results in the preparation of 9-amino nonanoic acid from the oxidative cleavage of oleonitrile or results in 11-amino undecanoic acid from the oxidative cleavage of the nitriles of lesquerolic acid or of vaccenic acid or of gondoic acid, preferably from the oxidative cleavage of the nitriles of vaccenic acid or of gondoic acid and more preferentially from the oxidative cleavage of the nitriles of vaccenic acid.

Finally, the invention also covers a process for the manufacture of a polyamide, which comprises the use of the process of the invention for preparing a heminitrile from a starting unsaturated fatty acid, followed by the hydrogenation of said heminitrile so as to obtain a corresponding ω-amino acid and, finally, the polymerization of said ω-amino acid so as to obtain said polyamide. Particularly preferably, said manufacture is carried out using corresponding starting fatty acids (or ester or oil derivatives of said fatty acids) which are of natural origin and from a renewable source. More particularly, according to this process, said polyamide is preferably polyamide 9 and said corresponding starting fatty acid is oleic acid or said polyamide is a polyamide 11 and said corresponding starting fatty acid is vaccenic acid or lesquerolic acid or gondoic acid, preferably vaccenic acid or gondoic acid and more preferentially vaccenic acid.

The process for preparing the heminitrile according to the invention is illustrated by the examples which follow.

The analysis methods used are set out in the text hereinafter.

The composition of the organic phase is analyzed by gas chromatography (GC) with an HP 5980 chromatograph.

The aqueous hydrogen peroxide content is analyzed using the Cefic Peroxygens H₂O₂AM7157 permanganate assay method. The iodine value is determined according to standard NF EN 14111.

The viscosity is measured at 40° C. with a Haake Viscotester VT550 with the NV measuring device.

### EXAMPLES 1 TO 12

The tests described below illustrate the reaction for oxidation of oleonitrile (ON) and also, by way of comparison, that of oleic acid (OA) and of methyl oleate (MO) by means of aqueous hydrogen peroxide, of varying the reaction parameters, i.e. the H₂O₂ content of the solution injected, and the injection molar ratios and flow rates, at a constant set temperature (70° C.) and under atmospheric pressure.

100 g of fatty compound and 1.1 g of tungstic acid (H₂WO₄; Merck 98%) are introduced into a 250 cm³ jacketed reactor comprising a mechanical stirrer, and then stirred and heated at 70° C., said temperature being maintained by circulation of thermostatic water. The aqueous hydrogen peroxide is then added in weight contents which are variable according to the tests, via a peristaltic pump at variable addition speeds according to the tests. The reaction is stopped after 6 h, the aqueous phase is separated for analysis. The remaining organic phase is washed several times with hot water until aqueous hydrogen peroxide has disappeared from the washing water.

The fatty substrates introduced come from the following sources:
oleonitrile (ON): Arkema with C_{16:0}: 3%, C_{18:0}: 9.7%, C_{18:1}: 84.7%, C_{18:2}: 1% (% by weight).
oleic acid (OA): Oleon Radiacid 0210 (C_{18:1}: 72%, C_{18:2}: 9% by weight)
methyl oleate (MO): Aldrich Grade Technique (C_{18:1}: 70% by weight)

The operating conditions and the results obtained are given in table 1 hereinafter, in which “molar ratio” denotes the H₂O₂/fatty compound molar ratio and NA denotes the presence (Y) or the absence (N) of nonanoic acid, characteristic of the cleavage of the molecule.

TABLE 1
operating conditions and results

| Exam- | | | | Flow | Initial | Final | |
|---|---|---|---|---|---|---|---|
| ple | | [H2O2] | Molar | rate | iodine | iodine | |
| No. | Substrate | (%) | ratio | g/min | value | value | NA |
| 1 | ON | 50 | 6 | 0.24 | 98 | 4 | Y |
| 2 | ON | 50 | 4 | 0.48 | 98 | 30 | Y |
| 3 | ON | 50 | 6 | 0.48 | 98 | 41 | Y |
| 4 | ON | 35 | 4 | 0.34 | 98 | 37 | N |
| 5 | ON | 50 | 4 | 0.48 | 98 | 32 | Y |
| 6 | MO | 35 | 1.8 | 0.128 | 91 | 74 | N |
| 7 | MO | 50 | 4 | 0.48 | 91 | 80 | N |
| 8 | MO | 50 | 4 | 0.48 | 91 | 87 | N |
| 9 | OA | 35 | 1.8 | 0.128 | 86 | 9 | N |
| 10 | OA | 50 | 2.7 | 0.5 | 86 | 9 | Y |
| 11 | OA | 35 | 1.8 | 0.256 | 86 | 33 | N |
| 12 | OA | 50 | 6 | 0.48 | 86 | 26 | Y |

The oleonitrile oxidation reaction makes it possible to substantially reduce the iodine value of the medium (see example 1) marking the disappearance of the double bonds (formation of diols or cleavage). The H₂O₂ concentration has an influence on the cleavage of the molecule treated (compare examples 1, 2, 3 and with example 4) resulting in heminitrile formation.

The methyl oleate oxidation reaction results only in a very low conversion of the double bonds regardless of the operating conditions. This oleic acid derivative is not therefore suitable for the formation of diacids by oxidative cleavage.

The oleic acid oxidation reaction allows a reduction in the iodine value of the medium (examples 9 to 12) and the formation of diacids, with a suitable H₂O₂ concentration.

### EXAMPLE 13

The oxidation of oleonitrile is an exothermic reaction which has an effect on the temperature of the reaction medium over time. The monitoring of this temperature, measured by thermocouple, makes it possible to have a better understanding of the oxidation process.

The experiment was carried out as follows by means of a reactor equipped with its stirrer used in the previous examples. In a first phase, the temperature of oleonitrile containing 1% by weight of H₂WO₄ catalyst is increased by means of a thermostatic bath at 70° C., and then two molar equivalents (2 mol per mole of nitrile) of H₂O₂ in solution at 50% are injected over the course of 40 minutes by means of a peristaltic pump. After these 40 minutes, in a second phase, the injection is stopped for 60 minutes. The solution is left to separate by settling out and the aqueous phase (with the majority of catalyst) is removed. Finally, at the beginning of the third phase, 1% by weight of H₂WO₄ catalyst is introduced and 4 molar equivalents (4 mol per mole of nitrile) of H₂O₂ in solution at 50% are injected over the course of 10 minutes by means of the peristaltic pump. The temperatures recorded in the reaction mixture throughout the reaction are given in table 2 below.

TABLE 2
temperature

| | Time (min) | | | | | | | | | |
|---|---|---|---|---|---|---|---|---|---|---|
| | 0 | 3 | 5 | 15 | 25 | 40 | 60 | 100 | 103 | 110 |
| T ° C. | 65.5 | 64.5 | 74.5 | 74 | 71 | 67.5 | 67 | 66 | 65.5 | 75 |

This example illustrates the advantage that can be gained from a sequence injection of catalyst and of H₂O₂.

### EXAMPLE 14

### Comparative

110 g of oleic acid (72% purity, containing 9% by weight of linoleic acid) and 1.1 g of tungstic acid are introduced into a 250 cm³ jacketed reactor comprising a mechanical stirrer, and then stirred and heated at 40° C. at atmospheric pressure. 77 g of aqueous hydrogen peroxide (49% by weight) are then added via a peristaltic pump at a speed of 0.92 cm³/min. The reaction is stopped after 24 h. The aqueous phase is separated and analyzed. The organic phase is washed several times with hot water until the aqueous hydrogen peroxide has disappeared from the washed water.

### EXAMPLE 15

### According to the Invention

92 g of oleonitrile (85% purity, containing 10% by weight of octadecanitrile, the nitrile of octadecanoic acid also known as stearic acid which is a saturated compound) and 0.9 g of tungstic acid are introduced into a 250 cm³ jacketed reactor comprising a mechanical stirrer, and then stirred and heated at 40° C. at atmospheric pressure. 77 g of aqueous hydrogen peroxide (at 49% by weight) are added via a peristaltic pump at a speed of 0.95 cm³/min. The reaction is stopped after 24 h. The aqueous phase is separated and analyzed. The organic phase is washed several times with hot water until the aqueous hydrogen peroxide has disappeared from the washed water.

The analysis results obtained are summarized in tables 3 to 6 below.

### H₂O₂ Contents

TABLE 3
H₂O₂ content

| | % H₂O₂ by initial weight | % H₂O₂ by weight after 24 h |
|---|---|---|
| Oleic acid | 49 | 45 |
| Oleonitrile | 49 | 32 |

The aqueous hydrogen peroxide concentration of the aqueous phase, initially 49% by weight, changes to 45% by weight after 24 h of reaction for oleic acid and 32% by weight for oleonitrile. The amount of aqueous hydrogen peroxide having reacted with the oleonitrile is substantially greater than that having reacted with the acid. This means that there is a greater progression of the substrate oxidation reaction, which is confirmed by the results of tables 4 to 6.

### Iodine Value:

The iodine value, which measures the concentration of double bonds, is determined before and after the reaction.

TABLE 4
iodine value

| | Initial iodine value | Iodine value after 24 h |
|---|---|---|
| | (g I₂/100 g of product) | (g I₂/100 g of product) |
| Oleic acid | 86 | 45 |
| Oleonitrile | 99 | 3 |

A clear decrease in the number of double bonds is noted in the case of oleonitrile. The reaction between the aqueous hydrogen peroxide and the oleonitrile, corresponding to the first phase of the process, progresses much more quickly than the reaction between the oleic acid and H₂O₂.

### GC (Gas Chromatography) Analysis

The gas chromatography analysis of the organic phase after reaction is carried out in order to determine the amount of nonanoic acid formed which shows that the oxidation has reached the cleavage stage.

TABLE 5
nonanoic acid analysis

| | Nonanoic acid (% by weight) |
|---|---|
| Oleic acid | 0.04 |
| Oleonitrile | 0.2 |

The formation of nonanoic acid, a product resulting from the cleavage of the fatty chain (second phase of the process), is observed. It is noted, in comparison with oleic acid, that the concentration of nonanoic acid resulting from the cleavage of oleonitrile is five times greater compared with that obtained with oleic acid.

### Viscosity Measurement

The viscosity η of the starting (initial) organic phase and also that obtained after 24 h of reaction (final viscosity) are measured using, respectively, rotational speeds of 245 s⁻¹ and 972 s⁻¹.

TABLE 6
viscosity

| | Rotational speed | Initial viscosity | Final viscosity |
|---|---|---|---|
| | (s⁻¹) | (mPa · s) | (mPa · s) |
| Oleic acid | 245 | 24 | 110 |
| Oleonitrile | 245 | 7 | 82 |
| Oleic acid | 972 | 19 | 90 |
| Oleonitrile | 972 | 7 | 70 |

The results obtained show that the viscosity increases during the reaction. However, it is noted that the viscosity of the organic phase obtained with oleic acid is greater than the viscosity obtained with oleonitrile, although the reaction between the aqueous hydrogen peroxide and the oleic acid is less advanced (see tables 4 and 5).

### EXAMPLES 16-20

### With Ammoniation of Oleic Acid and Oxidation of the Oleonitrile Obtained

Ammoniation of Oleic Acid in Order to Prepare Oleonitrile

Approximately 2000 g of fatty acid (oleic) are charged to a 4-liter predried glass reactor equipped with a mechanical stirrer, an electric heater, a dephlegmator, a condenser, a dry-ice trap and a system for introducing ammonia.

A catalytic feedstock of zinc oxide (0.0625% of the weight of fatty acid) is added.

The reaction medium is stirred, and then heated to 200° C. Gaseous ammonia is then introduced at a rate of 0.417 liters/min.Kg. The reaction medium is brought to 300° C. The introduction of ammonia is continued until the acid number of the reaction medium is less than 0.1 mg of KOH/g. The duration of the reaction is approximately 10 h.

At the end of the reaction, the reaction medium is cooled to 40° C. and the reactor is emptied.

The product is purified by distillation so as to obtain the oleonitrile, used hereinafter.

Oxidative Cleavage of Oleonitrile

The oleonitrile as prepared above and tungstic acid (H₂WO₄; Merck 98%) are introduced into a 250 ml (100 ml for No. 20) jacketed reactor equipped with a mechanical stirrer, and then stirred and heated at the temperature indicated in the table. The temperature is maintained by circulation of thermostatic water. The aqueous hydrogen peroxide is then added at various weight contents and at various speeds according to the tests, via a peristaltic pump. The reaction is stopped after the time indicated. The organic phase is separated and washed several times with hot water until the aqueous hydrogen peroxide has disappeared from the washing water. After drying of the organic phase under vacuum, the composition is determined by gas chromatography (GC). The GC analyses are carried out on an HP5890 series II instrument with an HP5 column and with an FID detector.

For the test of example 16, the aqueous hydrogen peroxide is added in the following way: 48 g of H₂O₂ at 70% by weight in water are added, at a constant flow rate over a period of approximately 45 min, to 150.4 g of oleonitrile containing 1.5 g of tungstic acid. After approximately 3 h, the aqueous phase is separated and a further 48 g of H₂O₂ at 70% by weight in water are added, with the same flow rate. This step is repeated again after 6 h, 21 h, 24 h and after 27 h, for an overall duration of the test of 42 h and with the overall addition of 288 g of H₂O₂ at 70% by weight in water. At each addition of aqueous hydrogen peroxide, 1.2 g of tungstic acid are added. Examples 19 and 20 were carried out with the reactor maintained under a stream of nitrogen.

The conditions of examples 16 to 20 are summarized in table 7 below.

TABLE 7
operating conditions of examples 16 to 20

| | | | Weight | | | | | |
|---|---|---|---|---|---|---|---|---|
| | | | H₂O₂ | H₂O₂ | | | | End of |
| | | Weight of | at X % | concentration | | Total | Weight | addition |
| Example | Composition | oleonitrile | in H₂O | as X % by | T | duration | H₂WO₄ | H₂O₂ |
| REF | of oleonitrile | (g) | (g) | weight | (° C.) | (h) | (g) | (min) |
| 16 | 1 | 150.4 | 288 | 70 | 70 | 42 | * | * |
| 17 | 1 | 80.5 | 132 | 50 | 70 | 6 | 0.8 | 90 |
| 18 | 1 | 80.7 | 92 | 70 | 70 | 6 | 0.8 | 82 |
| 19 | 2 | 79.9 | 92 | 70 | 70 | 24 | 8.0 | 131 |
| 20 | 2 | 39.6 | 45 | 70 | 80 | 24 | 4.0 | 121 |

* see text (procedure above)

Two compositions (composition 1 and composition 2) were used with regard to the oleic acid and the corresponding oleonitrile obtained. These compositions are presented in table 8 below.

TABLE 8
Compositions 1 and 2 of the samples of oleic
acid and of the corresponding oleonitrile

% by weight

| Component | Composition 1 | Composition 2 |
|---|---|---|
| C14:0 | 5.3 | 0.1 |
| C16:1 | — | 0.2 |
| C16:0 | 8.2 | 3.5 |
| C18:1 | 68.6 | 82.7 |
| C18:0 | 5.5 | 3.5 |
| C18:2 | 4.8 | 0.5 |
| C20:1 | 3.8 | 0.3 |
| C20:0 | 3.8 | 0.2 |

The molar yield results, expressed as % of moles of product considered relative to the number of moles of oleonitrile used at the start, are presented in table 9 below.

TABLE 9
molar yield results

*Molar yield (%)

| Example REF | Nonanoic acid | 8-cyanooctanoic acid |
|---|---|---|
| 16 | 45.8 | 43.3 |
| 17 | 10.4 | 9.3 |
| 18 | 12.4 | 5.9 |
| 19 | 42.4 | 40.8 |
| 20 | 46.9 | 53.3 |

*Molar yield = mols product/mols oleonitrile used at the start

### EXAMPLES 21 AND 22

### With Gondoic Acid

Raw Materials Used (Gondoic Acid): A and B
A) The process is carried out as in patent JP 9-278706 (paragraphs 22 to 28) using jojoba oil in order to obtain a 99% pure gondoic acid source;
B) a mixture in gondoic acid is produced starting from erucic rapeseed oil. After hydrolysis of the oil by saponification, then acidification, the fatty acids are distilled in order to isolate, on the one hand, the oleic acid-rich light acids and, on the other hand, the erucic acid-rich fraction. In doing so, a third fraction rich in gondoic acid is produced. This fraction thus contains 1% of palmitic acid (C16:0), 11% of oleic acid (C18:1), 12% of linoleic acid (C18:2), 50% of gondoic acid (C20:1), 3% of behenic acid (C20:0) and 12% of erucic acid (C22:1).

Ammoniation of Gondoic Acid in Order to Prepare the Corresponding Nitrile

Approximately 2000 g of fatty acid (A or B) are charged to a 4-liter predried glass reactor equipped with a mechanical stirrer, an electric heater, a dephlegmator, a condenser, a dry-ice trap and a system for introducing ammonia. A catalytic feedstock of zinc oxide (0.0625% of the weight of fatty acid) is added. The reaction medium is stirred, and then heated to 200° C. Gaseous ammonia is then introduced at a rate of 0.417 liters/min.Kg. The reaction medium is brought to 300° C. The introduction of ammonia is continued until the acid number of the reaction media is less than 0.1 mg of KOH/g. The duration of the reaction is approximately 10 h.

At the end of the reaction, the reaction medium is cooled to 40° C. and the reactor is emptied.

The product is purified by distillation in order to obtain the gondoic nitrile.

The nitrile obtained from sample A results in fewer heavy products than B. The distillation makes it possible to eliminate the heavy products which form during the conversion of the acid to nitrile, but also the residual amides formed.

Oxidative Cleavage of the Nitrile of Gondoic Acid

The nitriles resulting from the acids A or B and tungstic acid (H₂WO₄; Merck 98%) are introduced into a 250 ml (100 ml for No. 20) jacketed reactor comprising a mechanical stirrer, and then stirred and heated at the temperature indicated in the table. The temperature is maintained by circulation of thermostatic water. The aqueous hydrogen peroxide is then added at various weight contents and at various speeds according to the tests, via a peristaltic pump. The reaction is stopped after the time indicated. The organic phase is separated and washed several times with hot water until the aqueous hydrogen peroxide has disappeared from the wash water. After drying of the organic phase under vacuum, the composition is determined by gas chromatography (GC). The GC analyses are carried out on an HP5890 series II instrument with an HP5 column and with an FID detector.

For example 21 (nitrile A), the aqueous hydrogen peroxide is added in the following way: 15 g of H₂O₂ at 70% by weight in water are added, at a constant flow rate over a period of approximately 45 min, to 80 g of nitrile A containing 1.5 g of tungstic acid. After approximately 3 h, the aqueous phase is separated and a further 15 g of H₂O₂ at 70% by weight in water are added, with the same flow rate. This step is repeated again after 6 h, 21 h, 24 h and after 27 h, for an overall duration of the test at 42 h and with the overall addition of 90 g of H₂O₂ at 70% by weight in water. At each addition of aqueous hydrogen peroxide, 1.5 g of tungstic acid are added. The tests were carried out with the reactor under a stream of nitrogen. The test of example 22 with nitrile B is carried out under the same conditions as the test of example 21 (see table 10 below).

TABLE 10
conditions of tests 21 and 22

| | | | Weight | | | | |
|---|---|---|---|---|---|---|---|
| | | Weight | H₂O₂ at | | | | |
| | | of | X % in | H₂O₂ | | Total | Weight |
| Example | Gondoic | nitriles | H₂O | concentration X | T | duration | H₂WO₄ |
| REF | nitrile | (g) | (g) | (% by weight) | (° C.) | (h) | (g) |
| 21 | A | 80 | 90 | 70 | 70 | 42 | 9.0 |
| 22 | B | 80 | 90 | 70 | 70 | 42 | 9.0 |

The molar yield results are presented in table 11 below.

TABLE 11
molar yield results

Molar yield (%)

| Example REF | Nonanoic acid | 10-cyanodecanoic acid |
|---|---|---|
| 21 (nitrile A) | 38 | 36 |
| 22 (nitrile B) | 35 | 33 |

The molar yield is calculated relative to the gondoic nitrile initially present for 10-cyanodecanoic acid and relative to all the omega-9 fatty nitriles present in the feedstock, for nonanoic acid.

### EXAMPLE 23

### With Hydrogenation of the Heminitrile in Order to Obtain the Corresponding Amino Acid

### Heminitrile: 10-cyanodecanoic acid (or cyano-10-decanoic acid)

Example 21 is reproduced while continuing the oxidation with H₂O₂ and while renewing the aqueous phase with aqueous hydrogen peroxide at 70% and with catalyst every 3 hours for a period of 48 h. At the end of this phase, the aqueous phase is removed and the product recovered is first distilled under vacuum in order to remove the pelargonic acid which has formed, and then the product is recrystallized from acetic acid.

### Hydrogenation of the Heminitrile: Obtaining 11-aminoundecanoic acid

An Ru/SiC catalyst is introduced into a stainless steel autoclave with a capacity of 500 ml, equipped with an electromagnetic stirrer. A solution containing 2 g of 10-cyanodecanoic acid, obtained above in accordance with the invention, and of mixed solvent composed of 140 ml of ethanol and 140 ml of aqueous ammonia containing 28% by weight of ammonia, is introduced into the autoclave. After having flushed the reactor several times with nitrogen, the reactor is pressurized at 35 bar with hydrogen. The reactor is then heated to 110° C. and the stirring and the temperature are kept constant for 1.5 h. The reaction then no longer consumes hydrogen and the autoclave drops in temperature to 70° C., and then the pressure is reduced to atmospheric pressure and a colorless liquid is withdrawn. The solvent is then evaporated off under vacuum at approximately 60° C. and white crystals (1.2 g) of 11-undecanoic acid are recovered.

1. A process for synthesizing a heminitrile of formula CN—(CH₂)ₙ—COOH or of formula CN—R′—COOH, in which formulae n is between 4 and 13 (limits included) and R′ represents an alkylene radical comprising from 4 to 13 carbon atoms and from 0 to 2 double bonds,
with said synthesis being carried out using a compound of unsaturated fatty acid (including ester or glyceride) type of natural origin, corresponding to the formula
(R₁—CH═CH—[(CH₂)_{q}—CH═CH]ₘ—(CH₂)ᵣ—COO—)ₚ-G
in which formula:
R1 is H, or an alkyl radical having from 1 to 11 carbon atoms comprising, where appropriate, a hydroxyl function,
q is an index 0 or 1,
m and p are whole indices, m being 0, 1 or 2 and p being between 1 and 3 (limits included),
if p is 1, in this case, G is an H, an alkyl radical having from 1 to 11, carbon atoms, or a radical comprising two or three carbon atoms, bearing one or two hydroxyl function(s),
if p is 2, in this case, G is the residue of a diol or of glycerol bearing a hydroxyl function,
if p is 3, in this case, G is the residue of glycerol,
r is a whole index between 4 and 13 (limits included),
with it being possible for the C═C double bonds in said formula to be in cis or trans conformation and with said process comprising a first step of ammoniation of the compound of unsaturated fatty acid, ester or glyceride type, resulting in the corresponding unsaturated nitrile, which nitrile is subjected, in a second step, to an oxidative cleavage in two successive phases with the formation of intermediate compounds of vicinal diol type, using H₂O₂ as oxidizing agent, in at least one of the two phases, so as to result in said heminitrile.

2. The process as claimed in claim 1, wherein the heminitrile of formula CN—(CH₂)ₙ—COOH is obtained from a compound of unsaturated fatty acid type corresponding to the formula R₁—CH═CH—(CH₂)ᵣ—COOG, in which formula G is an H, an alkyl radical having from 1 to 11 carbon atoms, or a radical comprising two or three carbon atoms, bearing one or two hydroxyl function(s).

3. The process as claimed in claim 1, wherein the heminitrile of formula CN—R′—COOH is obtained from a compound of unsaturated fatty acid type corresponding to the formula (R₁—CH═CH—[(CH₂)_{q}—CH═CH]ₘ—(CH₂)ᵣ—COO—)ₚ-G.

4. The process as claimed in claim 1, wherein the first phase of the second step, resulting in the vicinal diols, is carried out by oxidation of a double bond or double bonds using H₂O₂ as oxidizing agent, in the presence of an oxidation catalyst.

5. The process as claimed in claim 4, wherein H₂O₂ is injected into the medium in an amount representing from 1 to 4 molar equivalents, i.e. from 1 to 4 mol of H₂O₂ per mole of unsaturated nitrile to be oxidized, more particularly in the form of an aqueous solution having an H₂O₂ content of between 30% and 70% (limits included) by weight (by mass).

6. The process as claimed in claim 4, wherein the reaction of said second step is carried out at a temperature of between 20 and 80° C. (limits included).

7. The process as claimed in claim 4, wherein the second phase of said second step, of oxidative cleavage of the diols, is carried out with O₂ as oxidizing agent for cleavage of the C—C bond between the vicinal hydroxyls, in the presence of a catalyst chosen from cobalt salts such as in the form of acetate (Co(Ac)₂.4H₂O), chloride or sulfate or salts of Cu, Cr, Fe or Mn, and also the catalysts used during the first phase, chosen from tungstic acid (H₂WO₄) and its sodium salt Na₂WO₄ and Co/W mixtures.

8. The process as claimed in claim 7, wherein the amount of O₂ introduced is at least equal to the stoichiometric amount of the reaction, the O₂/vicinal diol molar ratio preferably being between 3/2 and 100/1 (limits included).

9. The process as claimed in claim 4, wherein the second phase of oxidative cleavage of the vicinal diols is carried out with H₂O₂ as oxidizing agent for cleaving the C—C bond between the vicinal hydroxyls and with an H₂O₂/vicinal diol molar ratio of between 3/1 and 10/1 (limits included).

10. The process as claimed in claim 1, using two separate reactors for implementing the second step, wherein the effluent resulting from the first phase is subjected to a partial separation of the aqueous and organic fractions, allowing the partial elimination of the aqueous fraction and the recycling, at the top of the first-phase reactor, of a part of the organic fraction, representing from 1% to 10% by weight of said unsaturated nitrile.

11. The process as claimed in claim 1, wherein use is made of a single reactor with H₂O₂ as sole oxidizing agent for the two phases, the H₂O₂/nitrile molar ratio being between 4/1 and 10/1 (limits included).

12. The process as claimed in claim 1, wherein a sequential injection of the catalysts is carried out through the course of the reaction process.

13. The process as claimed in claim 1, wherein it uses oleic acid or lesquerolic acid or vaccenic acid or gondoic acid, of natural origin, as said unsaturated fatty acid.

14. The process as claimed in claim 1, wherein it comprises an intermediate step of metathesis of the nitrile resulting from said ammoniation step, so as to result in an unsaturated nitrile before the second step where said unsaturated nitrile is subjected to said oxidative cleavage so as to give said heminitrile, said metathesis being carried out with ethylene (ethenolysis), propylene, 1-butene or 2-butene.

15. The process as claimed in claim 1, wherein it comprises an intermediate step of ethenolysis of the nitrile resulting from said ammoniation step, so as to result in an ω-unsaturated nitrile, this being before the second step where said ω-unsaturated nitrile is subjected to said oxidative cleavage so as to result in said heminitrile.

16. The process as claimed in claim 1, wherein the nitrile used in the second step is oleonitrile or the nitrile of lesquerolic acid or the nitrile of vaccenic acid or the nitrile of gondoic acid.

17. The process as claimed in claim 1, wherein said unsaturated fatty acid is prepared in a prior step of said process, comprising the hydrogenation of a corresponding starting hydroxylated unsaturated fatty acid, so as to obtain the corresponding hydrogenated acid, said hydrogenation being followed by dehydration of said hydrogenated acid.

18. The process as claimed in claim 17, wherein said fatty acid is vaccenic acid and wherein said starting hydroxylated unsaturated fatty acid is ricinoleic acid and wherein said hydrogenated acid is 12-hydroxystearic acid (12-HSA).

19. The process as defined in claim 1, wherein it is part of a method of preparation of polyamide monomers, selected from ω-amino acids and/or the diamines and/or the diacids equivalent to said heminitrile and/or in that said use relates to the production of polyamides.

20. The process as claimed in claim 19, wherein said monomer is an ω-amino acid equivalent to said heminitrile and wherein said process comprises an additional step of hydrogenation of the nitrile function of said heminitrile and the conversion thereof into the corresponding amine function.

21. The process as claimed in claim 20, wherein said process is as defined in claim 16 and results in the preparation of 9-aminononanoic acid from the oxidative cleavage of oleonitrile or in that said process is as defined in claim 16 or 18 and results in 11-aminoundecanoic acid from the oxidative cleavage of the nitriles of lesquerolic acid or of vaccenic acid or of gondoic acid.

22. A process for the manufacture of a polyamide, wherein it comprises the use of the process as defined in claim 1 for preparing a heminitrile from a starting unsaturated fatty acid, followed by the hydrogenation of said heminitrile so as to obtain a corresponding ω-amino acid and, finally, the polymerization of said ω-amino acid so as to obtain said polyamide.

23. The process as claimed in claim 22, wherein said manufacture is carried out using corresponding starting unsaturated fatty acids (or ester or oil derivatives of said fatty acids) which are of natural origin and from a renewable source.

24. The process as claimed in claim 22, wherein said polyamide is:
polyamide 9 and said corresponding starting unsaturated fatty acid is oleic acid,
polyamide 11 and said corresponding starting unsaturated fatty acid is vaccenic acid or lesquerolic acid or gondoic acid.
